(19) United States
(12) Patent Application Publication
(10) Pub. No.: US 2016/0045632 A1
(43) Pub. Date: Feb. 18, 2016

(54) METHOD AND APPARATUS FOR DISPERSING AN ACTIVE SUBSTANCE

(71) Applicant: paragon ag, Delbrueck (DE)

(72) Inventor: Ralf MOENKEMOELLER, Bielefeld (DE)

(21) Appl. No.: 14/808,434

(22) Filed: Jul. 24, 2015

(30) Foreign Application Priority Data
Aug. 14, 2014 (DE) 102014012153.4

(51) Int. Cl. A61L 9/03

(52) U.S. Cl. CPC A61L 9/03

(57) ABSTRACT

An apparatus for dispersing an active substance has an active-substance reservoir from which active substance can be extracted and introduced into an interior space by an air stream. In order to achieve with as little expenditure as possible that the air stream has a temperature level which is optimum for extracting the active substance from the active-substance reservoir, it is proposed that the apparatus for dispersing an active substance has a mixing station, at which air streams having different temperatures are able to be mixed to the air stream so that the latter is able to be adjusted to an optimum temperature level for extracting the active substance, preferably between 19° C. and 24° C.

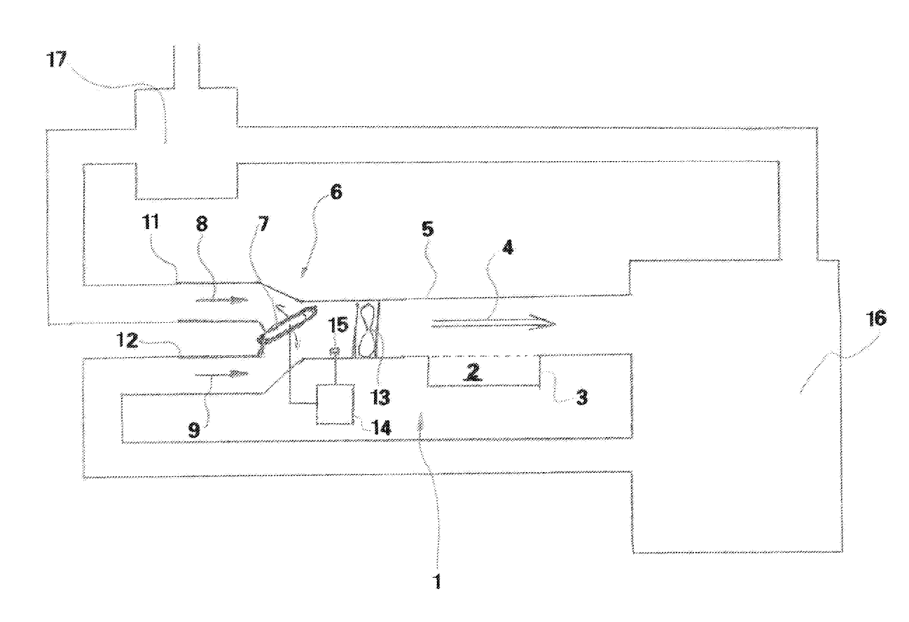

### FIELD OF THE INVENTION

The present invention relates to the dispersal of an active substance. More particularly this invention concerns a method of and apparatus for dispersing an active substance, such as a fragrance.

### BACKGROUND OF THE INVENTION

An active substance is typically dispersed from an reservoir from which active substance is able to be extracted and introduced into an interior space by an air stream, or respectively relates to a corresponding method for dispersing an active substance from an active-substance reservoir, in which an air stream is directed past the active-substance reservoir and the air stream receives active substance at the active-substance is reservoir.

The above-mentioned active substances are substantially fragrances that can be stored in various, for example solid or liquid, forms. The release into the interior space air takes place via the air stream. This air stream or a portion of this air stream is directed over surfaces of the active-substance reservoir and thus carries a portion of the active substances stored in the active-substance reservoir into the interior space.

From DE 195 31 518 A1 a method and an apparatus for fresh air neutralization are known, in which characteristics of the air stream are detected and used for a conditioning of the air stream. A fundamental problem in such methods and apparatuses is not solved here. Each active substance has an optimum operating temperature range. In this operating temperature range, the active substance has its defined characteristics. Active substances used as fragrances are soluble in air to a lesser extent at cold temperatures. The air occurring in the interior space is then possibly not perceptible in the correct intensity. At comparatively high temperatures, it is possible that the entire fragrance composition becomes disordered and is felt by occupants to be unpleasant.

In order to heat the temperature level of the air stream to the desired target value, the use of an electric heating system is known; in order to cool the temperature level of the air stream to a desired target value, the use of a so-called Peltier element is known. In the case of these known heating or respectively cooling methods, however, a comparatively high expenditure of electrical energy is necessary. Precisely in view of the fuel consumption in motor vehicles with internal combustion engines and the energy consumption in electric vehicles, this comparatively high expenditure of energy for a purely comfort feature, this being concerned with the fragrance present in the interior of the vehicle, is not acceptable.

### OBJECT OF THE INVENTION

Proceeding from the prior art described above, the invention is based on the problem of providing an apparatus for dispersing an active substance, in which the temperature control of the air stream flowing past the active-substance reservoir is able to be realized with a considerably lower energy consumption.

### SUMMARY OF THE INVENTION

This problem is solved as regards apparatus technology in that the apparatus for dispersing an active substance has a mixing station, at which air streams having different temperatures are able to be mixed to the air stream such that the latter is able to be adjusted to an optimum temperature level for extracting the active substance, preferably between 19° C. and 24° C. As regards method technology, this problem is solved in that the air stream is mixed from air streams having at least two different temperatures, and that the at least two air streams are mixed together to the air stream so that the air stream is adjusted to an optimum temperature level for extracting the active substance.

In a particularly advantageous automotive application, the apparatus for dispersing an active substance according to the invention is connected at the outlet side to the vehicle passenger compartment of a motor vehicle and the mixing station of the apparatus is connected at the inlet side to the vehicle passenger compartment and to a heating, ventilating and air-conditioning (HVAC) system of the motor vehicle, from which one of the two air streams is able to be fed to the mixing station. By corresponding adjustment of the mixture ratio between the air stream originating from the vehicle passenger compartment and the air stream originating from the HVAC system, the air stream flowing past the active-substance reservoir can be adjusted to the optimum temperature level for dispersing active substance from the active-substance reservoir.

Expediently, the apparatus for dispersing an active substance according to the invention has a temperature controller, by means of which the temperature level of the air stream at the outlet side of the mixing station is able to be regulated electronically or thermostatically.

The mixing station can expediently be embodied as a separate mixing module, if applicable with an integrated blower, or as an integrated module of the apparatus for dispersing an active substance.

In the automotive application, in the method according to the invention the air stream is directed from the outlet side of the apparatus for dispersing an active substance into a vehicle passenger compartment of a motor vehicle and an air stream fed from the vehicle passenger compartment and an air stream fed from a HVAC system of the motor vehicle are mixed to the air stream.

Expediently, the temperature level of the air stream that has been mixed together is regulated electronically or thermostatically.

In order to direct the air stream in the desired quantity past the active-substance reservoir, it is advantageous if the air stream is conveyed by a blower arranged downstream of a mixing station of the apparatus for dispersing an active substance.

### BRIEF DESCRIPTION OF THE DRAWING

The invention is explained in further detail below with the aid of an embodiment with reference to the drawings, the single FIGURE of which shows in a basic illustration an embodiment of the apparatus for dispersing an active substance according to the invention.

### SPECIFIC DESCRIPTION OF THE INVENTION

An active-substance reservoir 3 is provided in the embodiment shown in the single FIGURE of the apparatus 1 according to the invention for dispersing an active substance 2. In the active-substance reservoir 3, the active substance 2 is stored so that an air stream 4 that flows through a suitable duct 5 or the like past the active-substance reservoir 3 can take up or extract active substance 2 from the active-substance reservoir 3.

In addition, the apparatus 1 has a mixing station 6 where the air stream 4 is mixed together from—in the illustrated embodiment—two air streams 8, 9. An adjustable flap 7 serves to control with or without feedback the entry of the air stream 8 from a feed conduit 11 into the duct 5 and the entry of the air stream 9 out from a feed conduit 12 into the duct 5.

In addition, in the embodiment of the apparatus 1 according to the invention for extracting the active substance 2, shown in the single FIGURE, a blower 13 is provided that can be a fan or a ventilator that draws air from the feed conduits 11, 12 into the duct 4. The proportion of the volume flows of the air streams 8, 9 into the air stream 4 is variable as a function of the position of the flap 7.

In order to establish in the air stream 4 an optimum temperature level for extracting the active substance 2 from the active-substance reservoir 3 through the duct 5 past the active-substance reservoir 3, the apparatus 1 for extracting the active substance 2 has a temperature controller 14 associated with the mixing station 6. The temperature controller 14 is connected to a temperature sensor 15 that, viewed in the flow direction, is arranged downstream of the flap 7 in the duct 5 through which flows the air stream 4. When the temperature of the air stream 4 that passes the sensor 15 of the temperature controller 14 does not correspond to the desired target value that preferably lies between 19° C. and 24° C., the position of the flap 7 can be altered by the temperature controller 14 such that the relative proportions of the air stream 8 or of the air stream 9 in the air stream 4 is adjusted so that the air stream 4 has the desired temperature.

The active substances 2 stored in the active-substance reservoir 3 of the apparatus 1 for dispersing an active substance are frequently fragrances that can be stored in solid or liquid forms. The release of these active substances or respectively fragrances takes place via the air stream 4.

In the illustrated embodiment, the apparatus 1 for dispersing an active substance 2 is described by way of example in connection with a motor vehicle. The air stream 4, and with the air stream 4 the active substance 2, is introduced into a vehicle passenger compartment 16 by the duct 5. Here, the active substance or fragrance 2 ensures that the vehicle occupants in the vehicle passenger compartment 16 feel comfortable.

The mixing station 6 is connected to a heating, ventilating and air-conditioning (HVAC) system 17 of the motor vehicle via the feed conduit 11 through which the air stream 8 arrives at the mixing station 6. Accordingly, the air stream 8 has the temperature that the temperature-controlled air has on leaving the HVAC system 17. It is pointed out that the HVAC system 17 can of course be connected to the vehicle passenger compartment 16 via further conduits that are not illustrated in the FIGURE. Accordingly, in the mixing station 6 the air stream 8 fed from the HVAC system is mixed with the air stream 9 drawn from the vehicle passenger compartment 16 to the mixing station 6 so that the air stream 4 flowing past the active-substance reservoir 3 in the duct 5 has the desired temperature level.

The temperature controller 14 can regulate the temperature level of the air stream 4 at the outlet side of the mixing station 6 electronically or thermostatically.

The mixing station 6 of the apparatus 1 for dispersing an active substance 2 can be configured as a separate mixing module without or with the integrated blower 13. Of course, it is also possible to configure the mixing station 6 as an integrated module of the apparatus 1 for dispersing an active substance.

Motor vehicles where the apparatus 1 for extracting the active substance 2 is used for perfuming the vehicle passenger compartment 16 are usually standard equipment with the HVAC system 17 that was already mentioned above. For such HVAC systems 17, there are basically three different operating modes, namely a heating mode, a cooling mode and a recirculate mode.

In the heating mode, the vehicle passenger compartment 16 must be heated very quickly, so that a comfortable temperature is reached quickly, this temperature level typically being considered as comfortable by the vehicle occupants when it is generally in a range between 19° C. and 24° C. This is always dependent on the personal preference of the vehicle occupants in the vehicle passenger compartment 16. In the winter, with external temperatures of −10° C. in a thoroughly cooled motor vehicle, heating of the vehicle passenger compartment can require ten or more minutes.

In the heating mode, in the apparatus 1 the temperature level of the air stream 4 necessary for extracting the active substance 2 is achieved in that the air stream 8 flowing out of the HVAC system 17 can lie above 60° C. and is mixed with the air stream 9 that is recirculate to the mixing station 6 from the vehicle passenger compartment 16 and that is still cold. Through a suitable mixing ratio between the air stream 8 and the air stream 9 that is adjustable by the temperature controller 14, the ideal temperature level for the air stream 4 can be adjusted.

In the cooling mode, the vehicle passenger compartment 16 must be cooled in a very short time, so that a temperature level that is comfortable for the occupants is reached as quickly as possible. A comfortable temperature level is again the range which lies between 19° C. and 24° C. Here, also, this is dependent on the personal preferences of the vehicle occupants in the vehicle passenger compartment. Cooling of the vehicle passenger compartment in a vehicle that is heated in the sun to possibly 60° C. can require ten minutes or even more. In order to arrive at comparatively cool air in this cooling mode, the air stream 8 that arrives at the mixing station from the HVAC system 17 and flows out of the HVAC system 17 in the cooling mode can lie below 10° C. and is fed to the mixing station 6. In the mixing station 6, the air stream 8 is mixed with the air stream 9 that is recirculated to the mixing station 6 from the vehicle passenger compartment 16 that is still warm. Through a suitable mixing ratio between the air stream 8 and the air stream 9, the optimum temperature level is adjusted for the air stream 4 flowing past the active-substance reservoir 3 in the duct 5 of the apparatus 1 for extracting the active substance 2.

In the recirculate mode of the HVAC system 17, the temperature in the vehicle passenger compartment 16 has adjusted itself to the comfort range. In this case, the air stream 9 arriving from the vehicle passenger compartment 16 at the mixing station 6 forms the larger proportion of the air stream 4.

In the illustrated embodiment, the feed conduit 12 through which the air stream 9 arrives at the mixing station of the apparatus 1 for dispersing an active substance is connected to the vehicle passenger compartment 16.

I claim:

1. An apparatus for dispersing an active substance into an interior space, the apparatus comprising:
an active-substance reservoir from which an active substance can be extracted;
a mixing station;
a duct connecting the mixing station to the interior space and passing the reservoir;
means for supplying two air streams having different temperatures to the mixing station; and
means for varying the proportions of the two airstreams at the mixing station such that an air stream such that an air stream exiting the mixing station into the duct and passing over the reservoir has a temperature between 19° C. and 24° C.

2. The apparatus defined in claim 1, wherein the interior space is a motor-vehicle passenger compartment, the apparatus further comprising:
a recirculating conduit extending from the compartment to the mixing station and conveying one of the two air streams;
an HVAC system; and
a tempered-air conduit connected between the HVAC system and the mixing station and conveying the other of the two air streams to the mixing station.

3. The apparatus defined in claim 1, wherein the means for varying includes:
a temperature sensor in the duct downstream of the mixing station.

4. The apparatus defined in claim 1, wherein the mixing station of which is constructed as a separate mixing module.

5. The apparatus defined in claim 4, the separate mixing module of which has an integrated blower.

6. The apparatus defined in one of claims 1 to 3, wherein the mixing station is an integrated module of the apparatus for dispersing an active substance.

7. A method for dispersing an active substance from an active-substance reservoir into an interior space, the method comprising the steps of:
holding the active substance in an open reservoir;
mixing air from two air streams into a single combined air stream;
flowing the combined air stream past the active-substance reservoir and into the interior space while taking up the active substance with the combined air stream; and
varying the proportion of the air streams to establish in the combined air stream an optimum temperature.

8. The method defined in claim 7, wherein the interior space is a motor-vehicle passenger compartment, the method further comprising the steps of:
drawing air from the compartment as one of the two air streams; and
feeding air from an HVAC system as the other of the two air streams.

9. The method defined in claim 7, further comprising the step of:
detecting a temperature of the air in the combined air stream.

10. The method defined in claim 7, further comprising the step of:
conveying the combined air stream with a blower.
